(19) United States
(12) Patent Application Publication
(10) Pub. No.: US 2024/0308956 A1
(43) Pub. Date: Sep. 19, 2024

(54) LIPOPHILIC LITHIUM SALTS FOR DIRECT DISSOLUTION IN ORGANIC SCINTILLATORS

(71) Applicant: Lawrence Livermore National Security, LLC, Livermore, CA (US)

(72) Inventors: Natalia P. Zaitseva, Livermore, CA (US); Michael Joseph Ford, Livermore, CA (US); M Leslie Carman, San Ramon, CA (US)

(73) Assignee: Lawrence Livermore National Security, LLC, Livermore, CA (US)

(21) Appl. No.: 18/229,038

(22) Filed: Aug. 1, 2023

Related U.S. Application Data
(60) Provisional application No. 63/405,278, filed on Sep. 9, 2022.

(51) Int. Cl. C07C 309/17; C08K 5/00; C08K 5/42; C09K 11/02

(52) U.S. Cl. CPC C07C 309/17; C08K 5/0041; C08K 5/42; C09K 11/025

(57) ABSTRACT

A lithium (Li)-containing compound is a lipophilic Li salt, and each molecule of the lipophilic Li salt has at least one lipophilic endgroup. In addition, a scintillator material includes an organic liquid and a lipophilic Li salt, where the lipophilic Li salt is solubilized in the organic liquid. The scintillator material includes at least one fluorescent dye, the dye being effective to provide scintillation upon exposure to radiation. The scintillator material exhibits an optical response signature for thermal neutrons that is different than an optical response signature for fast neutrons.

### RELATED APPLICATIONS

This application claims priority to Provisional U.S. Application No. 63/405,278 filed on Sep. 9, 2022, which is herein incorporated by reference.

This invention was made with Government support under Contract No. DE-AC52-07NA27344 awarded by the United States Department of Energy. The Government has certain rights in the invention.

### BACKGROUND

Robust neutron radiation detectors with a large sensitive area, high volume, high detecting efficiency and a low cost means of making/using are important for the detection of neutrons in many areas such as nuclear nonproliferation, international safeguards, national security, scientific research, etc. In particular, for nuclear nonproliferation, fast and robust methods for the identification of special nuclear materials (SNM) are needed.

According to their energy, neutrons are typically divided in two major groups: thermal (low-energy) neutrons and fast (high-energy) neutrons. Detection of both types requires the separation of the neutron signatures from the always-present strong gamma radiation background. In common radiation detection practice, identification of both thermal and fast neutrons requires simultaneous use of two different types of detectors, one of which is based on mostly hydrocarbon-comprised scintillators (for fast neutron detection), and the other including scintillating materials loaded with thermal neutron capture reagents.

Efficient fluorescence combined with high hydrogen content of organic liquid scintillators enable the separation of neutron signatures from the gamma ray background determined by their pulse shape discrimination (PSD) properties. However, since the composition of organic scintillators is comprised of mostly hydrocarbons, traditional liquid scintillators can be used only for detection of fast neutrons, leaving undetected the large fraction of low-energy (thermal) neutrons that do not generate enough light in neutron proton (n,p) interactions. Lithium has very good properties for thermal neutron detection such that loading of organic scintillators with lithium-6 (⁶Li) nuclei is known as one of the ways to achieve additional sensitivity to thermal neutrons for producing light.

⁶Li possesses many desirable qualities as a neutron target. Among its important advantages are a reasonable capture cross section, relatively high photon yield from charged particles, and absence of gamma-rays in the final products resulting from a capture reaction ⁶Li+n^{o}=³H+α+4.8 MeV. The principal drawback, however, is very low or no solubility of highly polar ⁶Li-containing compounds in non-polar aromatic liquids needed for efficient scintillation. Early attempts to incorporate ⁶Li into liquid scintillators by simple dissolution of lithium salts of carboxylic acid did not lead to production of stable compositions. More success has been achieved with a different approach, in which a surfactant added to a liquid scintillator allows for the loading of an aqueous ⁶LiCl solution to produce a dynamically stable microemulsion.

The incorporation of ⁶Li into organic scintillators has been of interest for decades. In liquid scintillators, one approach to incorporate lithium-6 into organic scintillators involves the use of mixed liquids (e.g., toluene and methanol or dioxane and water). For example, lithium chloride is soluble in a mixture of dioxane that contains 20 wt. % water; however, there are disadvantages of dissolving lithium in a mixture of polar liquids because there is a large loss of light output. For example, the light yield of the organic scintillator is reduced by 65-70% upon addition of the salt. Organic salts of lithium may be more soluble in organic liquids due to the non-polar component of the salt. Thus, lithium propionate was used as it was soluble in mixtures of toluene and methanol. However, scintillators in these toluene/methanol mixtures had decreased light output as the content of methanol increased. Water and other polar liquids reduced the light output of these scintillators. Other efforts to load lithium into organic scintillators (e.g., into plastic scintillators) have required co-solvents to dissolve the lithium salt.

A successful technique to incorporate ⁶Li into organic liquid scintillator and plastic scintillator without including solubilizing agents, such as additional surfactants, water, hydrochloric acid, etc. remains elusive.

### SUMMARY

In one embodiment, a lithium (Li)-containing compound is a lipophilic Li salt, and each molecule of the lipophilic Li salt has at least one lipophilic endgroup.

In another embodiment, a scintillator material includes an organic liquid and a lipophilic Li salt, where the lipophilic Li salt is solubilized in the organic liquid. The scintillator material includes at least one fluorescent dye, the dye being effective to provide scintillation upon exposure to radiation. The scintillator material exhibits an optical response signature for thermal neutrons that is different than an optical response signature for fast neutrons.

In yet another embodiment, a plastic scintillator includes a polymer matrix and a lipophilic Li salt, where the lipophilic lithium salt is solubilized in the polymer matrix. The plastic scintillator includes at least one fluorescent dye, the dye being effective to provide scintillation upon exposure to radiation. The plastic scintillator exhibits an optical response signature for thermal neutrons that is different than an optical response signature for fast neutrons.

Other aspects and advantages of the present invention will become apparent from the following detailed description, which, when taken in conjunction with the drawings, illustrate by way of example the principles of the invention.

### BRIEF DESCRIPTION OF THE DRAWINGS

FIG. 1A is a plot of typical pulse shape discrimination (PSD) distribution measured with a PSD plastic without ⁶Li, according to one embodiment.

FIG. 1B is a plot of pulse shape discrimination (PSD) distribution measured with a PSD-capable organic scintillator loaded with ⁶Li, according to one embodiment.

FIG. 2 is a schematic drawing that outlines a possible synthetic route to obtain ⁶LiDOSS, according to one embodiment.

FIG. 3A is a graph depicting light output (LO) measured with different liquid scintillators loaded with ⁶LiDOSS, according to one embodiment.

FIG. 3B is a graph depicting pulse shape discrimination (PSD) measured with different liquid scintillators loaded with ⁶LiDOSS, according to one embodiment.

FIG. 4A is a graph depicting the light output (LO) of ⁶Li-aliphatic salts-loaded solutions, according to one embodiment.

FIG. 4B is a graph depicting the pulse shape determination (PSD) of ⁶Li-aliphatic salts-loaded solutions, according to one embodiment.

FIG. 5 is a plot of the dependence of light output (LO) and pulse shape discrimination (PSD) on ⁶Li concentration in scintillator liquids loaded with ⁶LiDOSS solution containing a single dye in combination with respective amounts of ⁶Li-aliphatic salt, according to one embodiment.

FIG. 6 is a plot of the dependence of light output (LO) and pulse shape discrimination (PSD) on ⁶Li concentration in organic scintillators containing solubilizing agents and a single dye with respective amounts of ⁶Li-aliphatic salt, according to one embodiment.

FIG. 7A is a graph of the comparison of the LO of ⁶Li-loaded scintillator liquids, according to one embodiment.

FIG. 7B is a graph of the comparison of the PSD FoM of ⁶Li-loaded scintillator liquids, according to one embodiment.

FIG. 8 shows a simplified layout of a radiation detection system, according to one embodiment.

### DETAILED DESCRIPTION

The following description is made for the purpose of illustrating the general principles of the present invention and is not meant to limit the inventive aspects claimed herein. Further, particular features described herein can be used in combination with other described features in each of the various possible combinations and permutations.

Unless otherwise specifically defined herein, all terms are to be given their broadest possible interpretation including meanings implied from the specification as well as meanings understood by those skilled in the art and/or as defined in dictionaries, treatises, etc.

It must also be noted that, as used in the specification and the appended claims, the singular forms “a,” “an” and “the” include plural referents unless otherwise specified.

For the purposes of this application, room temperature is defined as in a range of about 20° C. to about 25° C.

As also used herein, the term “about” denotes an interval of accuracy that ensures the technical effect of the feature in question. In various approaches, the term “about” when combined with a value, refers to plus and minus 10% of the reference value. For example, a thickness of about 10 nm refers to a thickness of 10 nm±1 nm, a temperature of about 50° C. refers to a temperature of 50° C.±5° C., etc.

It is also noted that, as used in the specification and the appended claims, wt. % is defined as the percentage of weight of a particular component relative to the total weight/mass of the mixture. Vol. % is defined as the percentage of volume of a particular compound relative to the total volume of the mixture or compound. Mol. % is defined as the percentage of moles of a particular component relative to the total moles of the mixture or compound. Atomic % (at. %) is defined as a percentage of one type of atom relative to the total number of atoms of a compound.

Unless expressly defined otherwise herein, each component listed in a particular approach may be present in an effective amount. An effective amount of a component means that enough of the component is present to result in a discernable change in a target characteristic of the ink, printed structure, and/or final product in which the component is present, and preferably results in a change of the characteristic to within a desired range. One skilled in the art, now armed with the teachings herein, would be able to readily determine an effective amount of a particular component without having to resort to undue experimentation.

The following descriptions refer to “plastics,” “liquids,” “solvents,” “optics,” “detector materials,” “detector compositions,” “scintillators,” etc. interchangeably. All such recitations shall be understood as referring to a material comprising one or more solvents and one or more fluors arranged in a manner so as to convey the ability to detect radiation of interest (e.g., thermal neutron) incident on the material.

The following descriptions refer to Lithium-⁶ (Li) containing materials. All such recitations of lithium containing material refer to material containing ⁶Li.

The description herein is presented to enable any person skilled in the art to make and use the invention and is provided in the context of particular applications of the invention and their requirements. Various modifications to the disclosed inventive aspects will be readily apparent to those skilled in the art upon reading the present disclosure, including combining features from various approaches to create additional and/or alternative approaches thereof.

Moreover, the general principles defined herein may be applied to other inventive aspects and applications without departing from the spirit and scope of the present invention. Thus, the present invention is not intended to be limited to the approaches shown but is to be accorded the widest scope consistent with the principles and features disclosed herein.

The following description discloses several preferred inventive aspects of lipophilic lithium salts for direct dissolution in organic scintillators and/or related systems and methods.

In one general embodiment, a lithium (Li)-containing compound is a lipophilic Li salt, and each molecule of the lipophilic Li salt has at least one lipophilic endgroup.

In another general embodiment, a scintillator material includes an organic liquid and a lipophilic Li salt, where the lipophilic Li salt is solubilized in the organic liquid. The scintillator material includes at least one fluorescent dye, the dye being effective to provide scintillation upon exposure to radiation. The scintillator material exhibits an optical response signature for thermal neutrons that is different than an optical response signature for fast neutrons.

In yet another general embodiment, a plastic scintillator includes a polymer matrix and a lipophilic Li salt, where the lipophilic lithium salt is solubilized in the polymer matrix. The plastic scintillator includes at least one fluorescent dye, the dye being effective to provide scintillation upon exposure to radiation. The plastic scintillator exhibits an optical response signature for thermal neutrons that is different than an optical response signature for fast neutrons.

A list of acronyms used in the description is provided below.

| | |
|---|---|
| C. | Celsius |
| DIPN | 2,6-diisopropylnaphthalene |
| DOSS | sodium 1,4-bis(2-ethylhexoxy)-1,4-dioxobutane-2-sufonate, |
| | dioctyl sulfosuccinate sodium salt |
| FoM | Figure of Merit |
| g | gram |
| IBA | Isobutyric acid |
| IVA | Isovaleric acid |
| keVee | kilo electron Volt electron equivalent |
| ⁶Li | Lithium-6 |
| LO | Light output |
| LS | Liquid scintillator |
| MeV | Mega electronvolt |
| mL | milliliter |
| ms | millisecond |
| PL | Photoluminescence |
| PROSPECT | Precision Reactor Oscillation and Spectrum |
| PSA | 3-phenylsalicyclic acid |
| PSD | pulse shape discrimination |
| Tx-100 | Triton X-100 |
| μm | micron |
| wt. % | weight percent |

Organic scintillators can detect thermal neutrons when loaded with compounds that facilitate capture reactions. For example, the capture reaction of lithium-6 produces an alpha-triton pair that provides a distinct signature enabling detection of thermal neutrons.

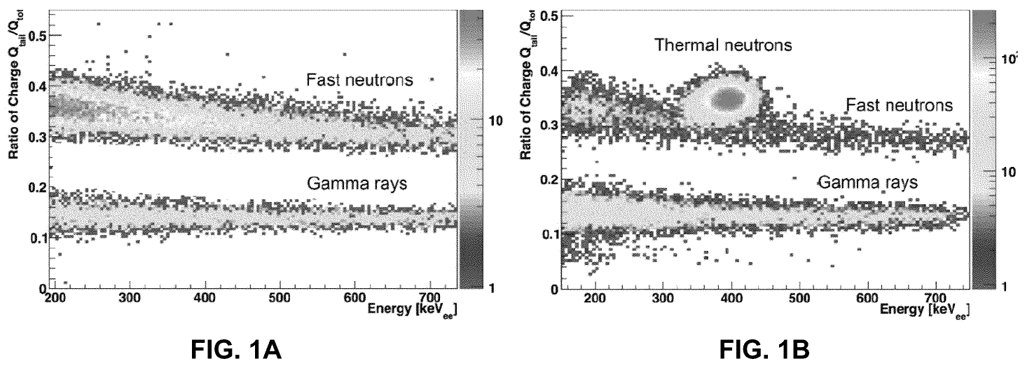

As illustrated in FIGS. 1A and 1B, incorporation of ⁶Li into a plastic composition leads to an additional signature of the monoenergetic α-particles produced as a results of thermal neutron interaction with ⁶Li atoms. FIG. 1A illustrates a typical PSD distribution measured with a PSD plastic without ⁶Li showing separated signatures of fast neutrons and gamma rays, according to one approach. FIG. 1B illustrates ⁶Li-loaded plastic producing “triple PSD” signatures of thermal neutrons, fast neutrons, and gamma rays, according to one approach. Thus, the “triple PSD” signature of ⁶Li-loaded plastic scintillators allows detection of both fast and thermal neutrons in one detection unit, without heavy moderation used in other thermal neutron detectors.

However, compounds that contain lithium-6 are not typically soluble in organic scintillators materials (e.g., organic liquids). A main challenge in preparation of ⁶Li-loaded plastics relates to the difficulties in incorporation of highly-polar Li-compounds in non-polar aromatic polymer matrices used for regular plastic preparation. Thus, the majority of current ⁶Li-containing organic scintillators are typically prepared with additives that reduce scintillation/PSD performance. For example, scintillator compositions having nano- or micro-particles of different Li-compounds in liquid or plastic matrices often have insufficient optical clarity and chemical instability due to inhomogeneous composition and structure.

Commercial production of organic scintillators with ⁶Li compounds has been problematic due to (1) difficult synthesis and purification procedures of the previously proposed ⁶Li compounds, (2) difficulties in scintillator preparation, and (3) insufficient light output (LO) caused by high self-absorption of the plastic composition components.

One of the most challenging aspects of using lithium-containing compounds in scintillators is that the lithium-containing compounds tend to be very polar while scintillation producing liquids are very nonpolar, and thus Li-containing compounds do not dissolve in such liquids. It would be desirable to develop a lithium molecule that is less polar and capable of dissolving in a nonpolar organic liquid without the addition of polar liquids and/or other solubility-enhancing additives.

Initial attempts included adding lithium to a commercial liquid scintillator solution such as EJ-309 liquid scintillator (LS) in an effort to find a simple way to obtain ⁶Li-loaded liquid scintillator. EJ-309 has a high scintillation LO and excellent PSD for fast neutrons. However, since lithium compounds are practically insoluble in pure aromatics, it was found that it became necessary to include solubilizing compounds for successful incorporation of ⁶Li into organic scintillation liquids such as EJ-309. In one example, an organic salt of lithium used in organic scintillators is lithium salicylate, which can facilitate capture of thermal neutrons and participate in scintillation. However, these studies found that polar solvents like dioxane are typically needed to dissolve lithium salicylate, and mixing dioxane with water is necessary to dissolve large amounts of lithium salicylate, and these processes of solubilizing lithium salicylate results in issues with long-term stability and precipitation of the salt.

Other attempts to solubilize ⁶Li salts into organic scintillators included adding surfactant as a solubilizing agent. For instance, the scintillator liquid used in reactors, such as the Precision Reactor Oscillation and Spectrum (PROSPECT) reactor, includes surfactants that are long molecules with hydrophilic and hydrophobic ends. For example, incorporation of surfactant-assisted water-dissolved ⁶LiCl into EJ-309 liquid can become possible in some cases due to the formation of small micelles with hydrophilic ends coupled to the polar water droplets, so that the opposite hydrophobic ends enable solubility of these micelles in a nonpolar aromatic medium.

These prior attempts also included the addition of surfactants in the absence of water by direct dissolution of some ⁶Li-salts of aromatic acids combined with common surfactants, such as, for example, Triton-X (Tx-100). In one instance, addition of 3 to 6 wt. % of Tx-100 allows for easy dissolution of several ⁶Li-salts of different salicylic acids, such as ⁶Li salicylate, ⁶Li-3-methylsalicylate, or ⁶Li-3,5-tertbutylsalicylate. Although some of these salts, like ⁶LiPSA (⁶Li salt of 3-phenylsalicylic acid) may be slightly soluble in aromatics, the addition of surfactant such as Tx-100 substantially increases solubility of the prepared mixtures that, at 0.1 wt. % of atomic ⁶Li, remain stable to precipitation during years of observation.

However, one of the disadvantages of using a surfactant in the absence of water is that only the very limited group of ⁶Li salts of salicylic acids benefit from the solubilizing properties of the surfactant, and which surfactant-⁶Li salt of salicylic acid system will render a successful result is unpredictable. Moreover, obtaining ⁶Li salts of salicylic acids involve complex purification and synthesis procedures. An additional problem with ⁶Li-salicylates is that being fluorescent compounds that emit at relatively long wavelengths (˜430 nm), they may work as inefficient secondary dyes quenching the final LO of other scintillation systems present that are emitting at shorter wavelengths, e.g., of 400-420 nm.

Moreover, further attempts to solubilize ⁶Li-containing compounds have included incorporating broader varieties of non-aromatic (aliphatic) ⁶Li salts using small additions of carboxylic acids as solubilizing agents. For example, this method has been applied for plastic scintillators loaded with short chain ⁶Li salts of butyric, methacrylic, and pivalic acids. Experimental studies have shown that most of these aliphatic salts can be dissolved in EJ-309 using 5-10 wt. % additions of isobutyric acid (IBA) relative to the EJ-309. Due to a lower aromatic fraction, plastics made with ⁶LiIVA have smaller PSD FoM compared to ⁶LiPSA, but both distributions corresponding to aromatic or aliphatic ⁶Li loads produce thermal and fast neutron signatures well separated from gamma rays, with high PSD FoMs and narrow thermal neutron peaks positioned at relatively high energy around 600-650 KeVee. Despite these good discrimination properties, however, both suggested methods suffer from the same problem such that there is a substantial loss of the LO in ⁶Li-loaded compositions compared to the initial EJ-309. As follows from measurements made with many EJ-309 samples loaded with ⁶Li, a typical decrease of the LO upon loading with different ⁶Li-salts, results in ˜35-40% LO loss, which is close to ˜30% as reported for the liquid scintillator used in the PROSPECT reactor. These findings indicate that ⁶Li and/or IBA (or other surfactant or additive) additions have an inherent effect on the LS resulting in LO loss. Thus, it is important to produce, discover, find, etc. a ⁶Li addition for, or composition alternative to, EJ-309 that has a diminished LO loss in ⁶Li-loaded liquid scintillators.

According to embodiments described herein, a Li-containing compound for organic scintillators may be a lipophilic Li salt since the lipophilic Li salt has increased solubility in organic liquids. A lipophilic Li salt is less polar and by its nature includes molecular structures that are lipophilic. A lipophilic molecule is defined as a molecule that tends to combine, dissolve, etc. in lipids, fats, oils, and nonpolar liquids (e.g., hexane, toluene, xylene, etc.). For the purposes of this disclosure, a liquid may be a solvent. In general, lipophilic salts have increased solubility in an organic liquid over an inorganic liquid, polar liquid, etc. In preferred approaches, a lipophilic Li salt demonstrates greater dissolution in an organic liquid over dissolution in a nonorganic liquid, e.g., a polar liquid such as water.

In some approaches, the lipophilic Li salt is soluble in an organic liquid in a range of greater than 1 g lipophilic Li salt per 100 mL of organic liquid up to 50 g of lipophilic Li salt per 100 mL of organic liquid. In some approaches, a lipophilic Li salt may have higher solubility in an organic liquid, for example a salt might be soluble up to around 300 g/100 mL of organic liquid. A preferable approach may include a solubility of a lipophilic Li salt in a range of greater than 1 g salt/100 mL of organic liquid to 100 g salt/100 mL or organic liquid.

In various approaches, lipophilic Li salts may act as surfactants themselves and/or exhibit properties of surfactants and promote solubility in organic nonpolar liquids. In some approaches, a surfactant having a structure characteristic of a lipophilic molecule, being a salt of a single charge cation, may be a preferred candidate for Li exchange for forming a lipophilic lithium salt. In one approach, a lipophilic salt that is soluble in an aromatic organic liquid is a salt of a metal where the metal cation may be replaced by Li.

One example of a lipophilic salt being a surfactant is the commercially available sodium lipophilic salt sodium 1,4-bis(2-ethylhexoxy)-1,4-dioxobutane-2-sulfonate (commonly known as dioctyl sulfosuccinate sodium salt, DOSS, or Aerosol OT). DOSS is readily soluble in nonpolar organic liquids. The lipophilic NaDOSS is soluble in polar liquids at about 1 g NaDOSS/100 mL of a polar liquid, but NaDOSS may be soluble in an organic liquid, e.g., toluene, at about 30 to 40 grams of NaDOSS/100 mL of an organic liquid. The lipophilic characteristics of the DOSS molecule promotes a higher solubility in organic liquids compared to polar liquids, such as water.

In one preferred approach described herein, the sodium of the DOSS molecule is replaced with lithium. In one application, a Li-6 DOSS (i.e., ⁶LiDOSS) may be promising for organic scintillators. As described herein, replacing the Na molecule with Li allows formation of a ⁶LiDOSS molecule that is soluble in organic liquids.

Conventional lithium salts (⁶Li salts) are highly polar and usually need a coating on the surface of the Li molecules in order to be dissolved in the aromatic liquid. For example, as discussed above, conventional ⁶Li salts need a solubilizing agent such as the surfactant Triton-X (Tx-100) to enable dissolution of the ⁶Li salts in the aromatic liquid. In one embodiment described herein, a lipophilic Li salt, e.g., ⁶LiDOSS, combines the surfactant property of being capable of dissolution in the aromatic liquid with the Li property. The lipophilic lithium salt preferably has properties of a surfactant and does not need additional solubilizing agents, liquids, surfactants, etc. for dissolution of the ⁶Li compound in the organic liquid. Accordingly, various embodiments include a ⁶Li compound in an organic liquid without additional solubilizing agents, liquids, surfactants, etc. added for dissolution thereof.

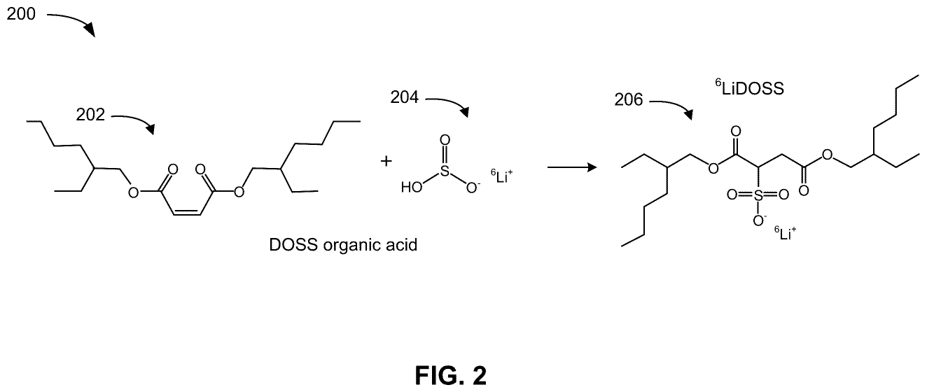

FIG. 2 illustrates a schematic drawing of an approach of synthesizing ⁶LiDOSS. A mechanism 200 of forming ⁶LiDOSS 206 may include a reaction between DOSS organic acid 202 and lithium sulfonate 204.

In one approach, ⁶LiDOSS may be synthesized by replacing sodium by ⁶Li in the molecule of NaDOSS commercially produced as Aerosol AT (AOT). Like Triton X-100 (Tx-100), NaDOSS has a hydrophilic head and long hydrophobic tails that facilitate formation of micelles used in preparation of stable water-oil emulsions. According to modeling and experimental studies, in the absence of water in non-polar systems, NaDOSS can form “dry” micelles with a core containing Na, S, and O atoms surrounded by the dense aliphatic surface layers that make it highly soluble in aromatic liquids. Due to the similarity in properties of sodium and lithium, formation of similar structures, that screen high polarity of the core, was expected for the ⁶Li analog of NaDOSS. In various approaches, production of ⁶LiDOSS demonstrates facile solubility in organic fluids at high concentrations up to about 50 wt. % or higher ⁶Li of total scintillator. In a preferred approach, solubility of ⁶LiDOSS in organic liquid may be up to 30 wt. %, and possibly higher.

Organic liquids for solubility ⁶LiDOSS may include an aromatic compound (e.g., an aromatic liquid, aromatics, etc.). Examples of organic liquids include toluene, styrene, xylene, 2,6-diisopropylnaphthalene (DIPN), etc. In preferred approaches, organic liquids include organic compounds that provide efficient scintillation performance. In some approaches, a liquid scintillator includes a water-based liquid. For instance, in some approaches a polar compound may be added to a liquid scintillator not for solubility of the lithium salt, but to dilute the scintillating medium in a polar compounds, which could reduce costs and introduce alternative detection capabilities. In one example, a dispersed liquid scintillator in water may include ⁶Li-lipophilic salt in an organic liquid that includes additional surfactant and water. A water-based liquid scintillator is advantageous for large volume detectors because water is less expensive than an organic scintillator. Other advantages of a water-based liquid scintillator include the possibility of reduced flammability (i.e., safer) and alternative modes of detection (e.g., Cherenkov detection, which relies on a specific interaction of nuclear particles with matter).

In preferred approaches, scintillating dyes may be dissolved in an organic liquid such as DIPN containing ⁶Li-lipophilic salt thereby allowing PSD of gamma rays, neutrons, and thermal neutrons. Alternatively, these lipophilic lithium salts may be directly dissolved in commercially available liquid scintillators like EJ-309 without significant reduction in scintillating performance. For example, the ⁶Li-lipophilic salt-loaded EJ-309 scintillators have a LO that is about 90% of the LO of EJ-309 without lithium and a PSD figure of merit (FoM) about 3.9. In some approaches, an organic scintillator loaded with ⁶Li-lipophilic salt may have a PSD FoM in a range of about 2 up to about 4.

Lipophilic Li salts may include molecular structures having a large polar functional group, e.g., a sulfonate group, which allows formation of coupled nonpolar, lipophilic units, e.g., large hydrocarbon chains, to the molecule. In one approach, each molecule of the lipophilic Li salt has at least one lipophilic end group. In a preferred approach, each molecule of the lipophilic Li salt has at least two lipophilic end groups. For example, lipophilic salts may include alkylsulfosuccinates (e.g., dialkylesters of sulfosuccinic acid), petroleum sulfonates, dinonyl naphthalene sulfonates, polycyclic aromatic hydrocarbon, alkylsucciniate (e.g., octyl succinate), etc.

Various methods may be employed to produce ⁶LiDOSS. Bis(2-ethylhexyl) maleate reacts with a metal bisulfite to form DOSS. Alternatively, the commercially available DOSS, in the form of sodium (Na) DOSS or possibly potassium DOSS may be obtained. The cation, Na⁺, K⁺ of the molecule may be exchanged with Li⁺.

In one approach of cation exchange, commercially available NaDOSS may be added to a strong acid in an organic liquid (e.g., methanolic HCl, water:methanol, etc.). NaCl precipitates, and the organic acid of DOSS can be obtained. This organic acid may undergo an acid-base reaction with a strong base of the metal of interest to form the final MDOSS (where M is the metal).

Another possibility is to use an ion exchange resin to form the organic acid of DOSS, followed by the acid-base reaction to obtain MDOSS. This process is amenable to produce ⁶LiDOSS with lithium-6 (⁶Li). An excess of ion exchange resin may be mixed with DOSS in deionized water for an appropriate duration of time, e.g., overnight. Preferably the ion exchange resin is specific for ion exchange reactions with sodium to be replaced with hydrogen. For example, AG® 50 W-X8 Cation Exchange Resin, hydrogen form, obtained commercially from Bio-Rad may be used. The ion exchange resin may be removed by filtration which produces a primarily organic acid conjugate, the organic acid of the original sodium lipophilic salt.

A Li base is then introduced to the organic acid of the lipophilic salt. An excess of a strong Li base, e.g., ⁶Li₂CO₃, ⁶LiOH, etc. may be added and mixed at an elevated temperature for an appropriate duration of time for essentially complete conversion of the organic acid to a lipophilic lithium salt. Water may be removed by evaporation. Dissolution of the ⁶LiDOSS in toluene and filtration of the product allows the removal of excess lithium base, e.g., ⁶Li₂CO₃, ⁶LiOH, etc. The ⁶LiDOSS (in toluene) may be dried over magnesium sulfate, filtered, and isolated by removing toluene.

In one approach the lipophilic Li salt is lithium 1,4-bis(2-ethylhexoxy)-1,4-dioxobutane-2-sulfonate (⁶LiDOSS). In various approaches, the lipophilic lithium salt is solubilized in organic liquids, e.g., organic liquid scintillator, without the addition of a solubilizing material typically included for adding ⁶Li to scintillator material. It is preferable to not include solubilizing agents in a scintillator material because the solubilizing agent, e.g., water, polar liquids, organic acid, additional surfactant, etc. reduce light output of the scintillator and thus reduce scintillation performance.

According to one embodiment, a scintillator material includes an organic liquid and a lipophilic Li salt, where the lipophilic Li salt is solubilized in the organic liquid. The scintillator material includes at least one fluorescent dye being effective to provide scintillation upon exposure to radiation. The scintillator material exhibits an optical response signature for thermal neutrons that is different than an optical response signature for fast neutrons.

In some approaches, the scintillator material is a liquid scintillator. In various approaches, lipophilic Li salts may be dissolved in organic liquids such as xylene, linear alkyl benzenes, organic solutions for scintillators, etc.

In one approach, lipophilic Li salt may be added to an organic scintillators that comprises a primary dye, e.g., 2,5-diphenyloxazole and a secondary dye, e.g., Exalite (e.g., E404), 1,4-Bis(2-methylstyryl)benzene (bis-MSB), etc., dissolved in an aromatic liquid, e.g., 2,6-diisopropylnaphthalene. In one approach, only a single fluorescent dye may be present in the scintillator material. In one approach, the scintillator material may include two fluorescent dyes being effective to provide scintillation upon exposure to radiation. In some approaches, the scintillator material may include more than two fluorescent dyes being effective to provide scintillation upon exposure to radiation.

Alternatively, in one approach, the organic liquid is a scintillator. For example, lipophilic Li salts may be directly dissolved in commercially available organic scintillators, e.g., EJ-309.

In one embodiment, a solubilizing agent is not present in the scintillator material. For the purposes of this disclosure, a solubilizing agent may include a polar liquid, an added surfactant, an acid, etc. In one approach, a scintillator material loaded with a lipophilic Li salt, such as ⁶LiDOSS, does not include acid, e.g., HCl, additional surfactants, polar liquids, solubility agents, etc. The absence of an acid in the scintillator composition promotes a less corrosive interface of the scintillator, and/or the equipment, containers, etc. in contact with the ⁶Li-loaded scintillator. In one approach, the lipophilic lithium salt may be a surfactant, and a second surfactant is not present in the scintillator material. Moreover, the absence of additional surfactant in the scintillator loaded with lipophilic lithium salt removes contamination of the scintillator with micelles that are typically formed in ⁶LiCl loaded scintillators that include acid and additional surfactants (e.g., Triton-X) and/or water in order to dissolve the ⁶LiCl in the liquid scintillator.

The scintillator loaded with lipophilic Li salt may not contain any polar liquid, e.g., water. In a preferred approach, a polar liquid is not present in the scintillator material loaded with lipophilic Li salt, i.e., the scintillator material is essentially free of polar liquid, e.g., water. Moreover, the polarity of ⁶LiDOSS is reduced relative to other lithium salts, for example, larger concentrations of ⁶Li such as about 0.3 wt. % to 0.1 wt. % may be dissolved in scintillator liquid.

The yield and performance of lipophilic Li salts are assessed for desired properties in organic scintillators. Li-based compounds in organic scintillators are tested for presence of Li, effect of light output of the organic scintillator, etc. The presence of lithium was confirmed by testing organic scintillators. In addition, chemical analysis confirmed the presence of ⁶Li in the organic scintillator.

The presence of lithium-6 (⁶Li) is confirmed by the presence of a thermal neutron spot at around 670 keVee. This approach to producing organic scintillators with pulse shape discrimination capabilities is promising for advanced detection capabilities.

Lipophilic Li salts allow the detection of thermal neutrons. In some approaches, detection of thermal neutrons is possible with a composition of the scintillator material having ⁶Li present in a range of 0.1 to 0.3 wt. % of ⁶Li. In one approach, using the PROSPECT reactor, a scintillator material may preferably have a concentration of 0.1 wt. % of ⁶Li which corresponds to about 7 wt. % of the ⁶LiDOSS. In other approaches, the range of ⁶Li in wt. % of scintillator material may be defined by a specific application, e.g., a specific detector. Increasing concentration of ⁶Li in the scintillator material increases efficiency of thermal neutron detection, but may decrease light output. In preferred approaches, the scintillator material is configured to have a relative light output upon scintillation that is at least 90% the light output of the scintillator material without the lithium-containing compound, e.g., lipophilic Li salt.

According to various embodiments, the Li-loaded scintillators formed with lipophilic Li salts demonstrate a light output nearly equivalent to organic scintillators without lithium. With no need for solubilizing acid and due to the decreased polarity of ⁶Li in the molecule, the addition of ⁶LiDOSS to the organic liquid scintillator produces less drop of LO or PSD compared to other compositions. In one approach, a lithium-loaded EJ-309 demonstrated >90% of the light output compared to the light output of EJ-309 without lithium. The addition of ⁶LiDOSS to the EJ-309 may cause within a 10% loss of light output. For comparison, liquid scintillators loaded with conventional lithium salts (e.g., ⁶LiCl) demonstrate a light output loss of 30% to 50% compared to liquid scintillators loaded with ⁶LiDOSS because the ⁶LiCl-loaded liquid scintillator includes polar additives for solubility and these polar additives reduce performance (e.g., light output). Without wishing to be bound by any theory, this phenomenon may relate to the low polarity of ⁶LiDOSS, confirming that in previous compositions, that PPO, for example, may be selectively affected by polar additions of acids and ⁶Li-salts.

Moreover, in the absence of such additions, ⁶LiDOSS-loaded EJ-309 also shows the highest FOM, largely determined by PSD properties of PPO less affected in this case by the ⁶Li addition. It should be added here that the high solubility of ⁶LiDOSS also allows for dissolution of larger concentrations of ⁶Li.

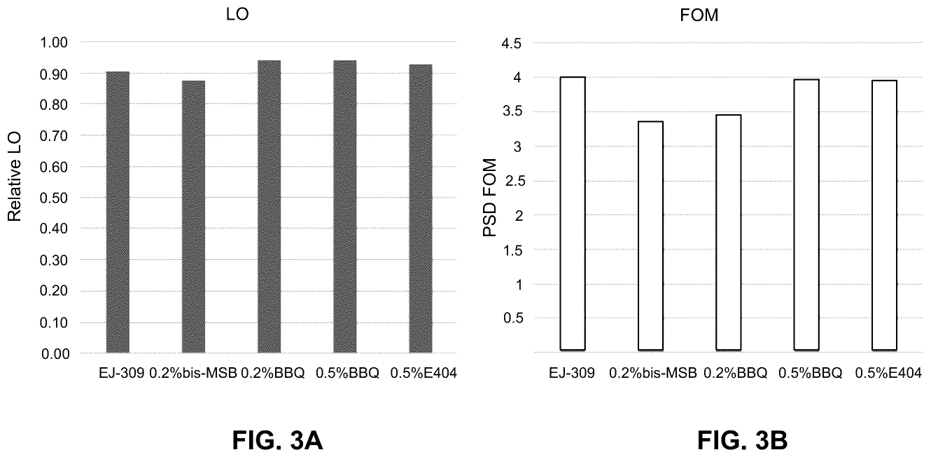

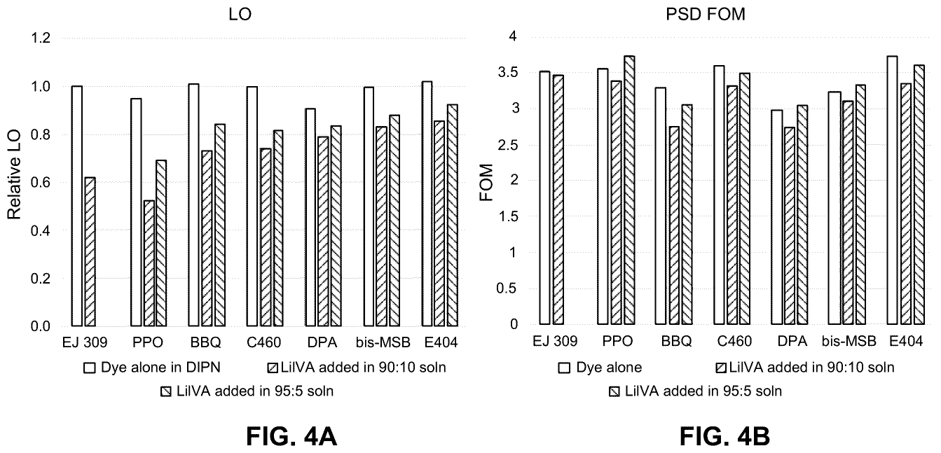

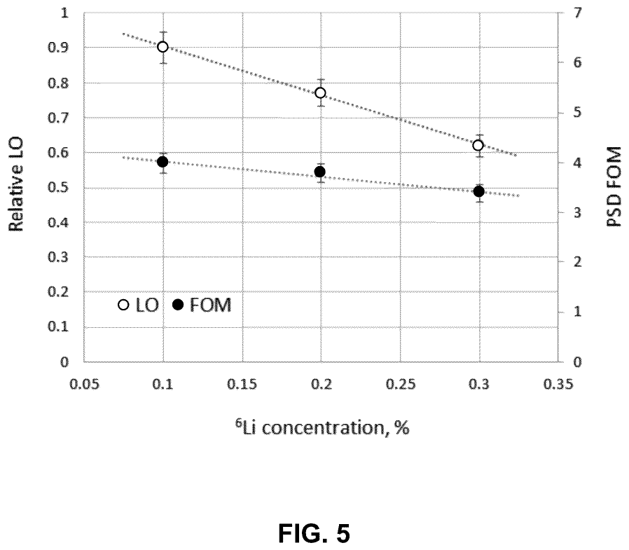

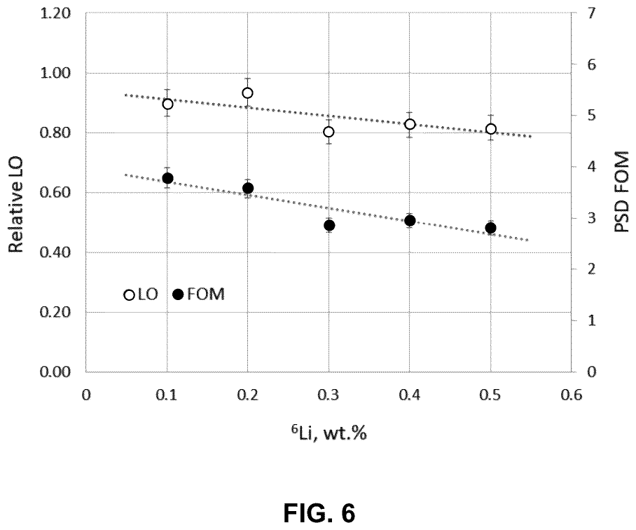

In some approaches, the solubility of the compound may be up to 40 wt. % without any additional dyes. In studies of long-term stability, solubility of the compound in DIPN may be about 20 wt. %, and loading to 0.3 wt. % of atomic ⁶Li may be problematic, but solutions containing 0.2 wt. % of atomic lithium are stable to long-term precipitation (FIG. 5). The higher concentration of ⁶Li leads to the decrease of the LO (FIG. 6). This drop may be attributed to a self-absorption or dilution by the non-aromatic fraction of ⁶LiDOSS. However, a remarkable property is that with less than 15% LO loss, and preferably less than about 10% LO loss (FIG. 3A), the ⁶LiDOSS allows for preparation of EJ-309-based LS with highest PSD among all tested composition (compare to FIG. 4B). In one approach, a scintillator material may be configured to have a relative LO upon scintillation that is at least 85% of the light output of the scintillator material without the Li-containing compound. In a preferred approach, a scintillator material may be configured to have a relative LO upon scintillation that is at least about 90% of the light output of the scintillator material without the Li-containing compound. However, the high performance combined with the simplicity of preparation that does not require addition of acids, which may cause long-term corrosive effects in large-volume liquid scintillators, makes ⁶LiDOSS-loaded EJ-309 one of the promising liquid scintillators for further development.

According to various embodiments, liquid scintillators loaded with lipophilic lithium salts demonstrate higher LO in both a small scale, in which volumes are approximately 3 mL based on a (Ø2 cm×1 cm) quartz cuvette, and a large scale, in which volumes are approximately 103 mL based on a (Ø5 cm×5 cm) quartz cuvettes. In some preferred approaches, ⁶LiDOSS-loaded EJ-309 LS may be more suitable for practical, large volume preparations.

In one embodiment, a plastic scintillator may include a polymer matrix and a lipophilic ⁶Li salt where the lipophilic ⁶Li salt is solubilized in the polymer matrix. In one approach, a plastic matrix material may be constructed, at least in part, of the cured organic liquid and the Li-containing compound, such that the Li-containing compound is a lipophilic Li salt, and each molecule of the lipophilic Li salt has at least two lipophilic groups. The plastic scintillator may include at least one fluorescent dye, the dye being effective to provide scintillation upon exposure to radiation. The plastic scintillator may exhibit an optical response signature for thermal neutrons that is different than an optical response signature for fast neutrons. In one approach, the plastic scintillator is a solid.

In one approach, a lipophilic ⁶Li salt may be dissolved a liquid monomer (e.g., plastic matrix material) before curing to form a plastic scintillator. In one approach, the lipophilic ⁶Li salt may be dissolved in an organic liquid that is configured to be cured into a plastic matrix material. In various approaches, plastic matrix materials may include, but are not limited to, aromatic polymers (e.g., polystyrene, polyvinyltoluene, etc.), non-aromatic polymers (e.g., methyl methacrylate, polydimethylsiloxane, other siloxane co-polymers, etc.), etc. In one approach, the plastic matrix material may be constructed at least in part of a cured organic liquid and the lipophilic ⁶Li salt.

### Experiments

EJ-309 and di-isopropylnaphthalene (DIPN) are standard commercial products that can be purchased from Eljen Technology. Most scintillation dyes, such as exalite E404 (1,4-bis(9,9-diethyl-7-(tert-pentyl)-9H-fluoren-2-yl)benzene), bis-MSB (1,4-bis(2-methylstyryl)benzene), C460 (7-diethylamino-4-methylcoumarin), and BBQ (4,4′″-bis[(2-butyloctyl)oxy]-1,1′:4′,1″:4″,1′″-quaterphenyl) were purchased from Exciton/Luxottica. PPO (2,5-diphenyloxazole), DPA (9,10-diphenylanthracene), BP (biphenyl) and Tx-100 (Triton-100) were produced by Sigma-Aldrich. ⁶Li-salts used for preparation of liquid scintillators were synthesized using reactions between ⁶LiOH and carboxylic acids, among which IBA (isobutyric acid) and IVA (isovaleric acid) were used as received.

Anhydrous ⁶LiOH was synthesized by reacting ⁶Li metal in a 1:1 solution of methanol and water followed by evaporating the liquids. The resulting ⁶LiOH H₂O was dissolved in methanol which was then evaporated under vacuum at 150° C. for 4 hours to give anhydrous ⁶LiOH. In another approach, ⁶Li₂CO₃ (National Isotope Development Center) was used as received instead of ⁶LiOH. ⁶Li-salts were synthesized by titrating corresponding acids with ⁶LiOH in methanol to pH=6 and evaporating the liquid. The resulting crude materials were washed with anhydrous diethyl ether to remove excess acids and dried on a Schlenk line at 90° C. under nitrogen for four hours to remove residual liquids.

⁶LiDOSS was produced using an adjusted method to load DOSS with ⁶Li. ⁶LiDOSS (⁶Li-1,4-bis(2-ethylhexoxy)-1,4-dioxobutane-2-sulfonate) was prepared in an excess ion exchange resin (30 g) from analogous sodium salt NaDOSS (commonly known as dioctyl sulfosuccinate sodium, or Aerosol OT) purchased from Aldrich. NaDOSS was added to methanolic HCl to precipitate NaCl and to obtain organic acid that was stirred with deionized water overnight to remove excess of ion resin by filtration. Excess ⁶Li carbonate (3.3 g) was added and mixed at 90° C. overnight to conduct acid-base reaction that forms the final ⁶LiDOSS. The excess ⁶Li carbonate was removed by dissolving the ⁶LiDOSS product in toluene and filtering. The product in toluene may be dried over magnesium sulfate and filtered. The product was isolated by removing toluene.

Majority of tested compositions were prepared with ˜0.1 wt. % of atomic ⁶Li, although a possibility of higher concentrations was also tested for some efficient formulations. For liquid preparations, solid constituents weighed in vials or glass jars at ambient condition were vacuumed and transferred into the nitrogen-filled glove box, where liquid components (EJ-309, DIPN, Tx-100, and IBA) were added for dissolution. Solutions were left to stirred overnight to reach equilibrium, then transferred into the quartz cuvettes, sealed, and removed from the glovebox. Unless indicated otherwise, measurements were made in small cylindrical cuvettes of Ø2 cm×1 cm in dimensions. For approximate evaluation of the attenuation, solutions of interest were also prepared and measured at larger scale in 2″×2″ (Ø5.1 cm×5.1 cm) quartz cuvettes.

Photoluminescence (PL) spectra were measured using a Horiba Jobin Yvon Fluoromax-4 spectrometer equipped with a 450 W Xe lamp in reflectance mode. Scintillation responses were characterized by wrapping the cuvettes in Teflon tape leaving open one flat window which was coupled using optical grease to a Hamamatsu R6231-100-SEL PMT. Signals collected from the PMT were recorded using a 14-bit high resolution CompuScope 14200 waveform digitizer at a sampling rate of 200 MS/s. Light output was evaluated from the ¹³⁷Cs gamma-ray response for which the 500 keVee location was defined by the 50% position of the Compton edge.

Comparison of the LO for different compositions was made relative to the small (Ø2 cm×1 cm) EJ-309 standard sample, LO of which was taken as a unity. Neutron/gamma PSD distributions were obtained using a ²⁵²Cf source moderated by 5.1 cm of lead and one inch (2.54 cm) thick high-density polyethylene. Resulting waveforms were integrated over two-time integrals corresponding to the total charge (Qₜₒₜₐₗ) and the delayed component of the signal (Qₜₐᵢₗ), respectively. The ratio R=Qₜₐᵢₗ/Qₜₒₜₐₗ indicated whether an event was likely produced by a neutron (large R) or a gamma-ray (small R). Quantification of the PSD was accomplished by calculating standard figures of merit (FoM)

$$FoM = \frac{〈 n 〉 - 〈 γ 〉}{{FWHM}_{n} + {FWHM}_{γ}}$$

where <n> and <γ> are the centroids of the neutron or gamma distributions, and FWHMn and FWHMγ are the full widths at half maximum of the neutron and gamma-ray distributions, respectively. For liquids without ⁶Li, fast neutron/gamma-ray PSD was evaluated in a regular way by calculation of FoM over the energy range 450-510 keVee. With ⁶Li-containing liquids, FoM was obtained by fitting the distributions over the energy region ±2.5σ around the centroid of the thermal neutron spot to a Gaussian function. It should be noted that because of the random effect of oxygen and other effects related to the experimental setup, LO and PSD of same size and composition prepared in different batches could show variations of about 5%. To avoid inconsistency in the final conclusions, each composition was prepared and measured in 2-4 individual samples, to select the highest parameters.

### Properties of Liquid Scintillators

Based on the molecular weight of the compound, obtaining 0.1 wt. % of ⁶Li included a relatively large addition of 7.1% wt. of ⁶LiDOSS. With no need for solubilizing acid and due to the decreased polarity of ⁶Li in the molecule, this addition produces less drop of LO or PSD compared to other compositions. As illustrated in FIG. 3A, addition of ⁶LiDOSS to single-dye solutions of bis-MSB, BBQ, or E404 leads to a decrease of only 6 to 10% relative LO. The relative LO values correspond to the LO of EJ-309 loaded with each dye in combination of ⁶LiDOSS compared to the unloaded EJ-309. The results depicted in FIG. 3A indicate that ⁶Li-loaded LSs may be prepared with a LO decrease not exceeding 6% compared to the unloaded EJ-309 standard.

For comparison, LSs containing aliphatic ⁶Li salts with the solubilizing agent isobutyric acid (IBA) (FIG. 4A) have diminished LO than the LSs containing ⁶LiDOSS (FIG. 3A). As illustrated in FIG. 4A, LSs include EJ-309 and single dye solutions in combination with aliphatic salt isovaleric acid (⁶LiIVA) dissolved in a 90:10 ratio mixture or a 95:5 ratio mixture of EJ-309 and IBA. The ⁶Li-loading produced the highest drop, nearly 40%, of the LO in PPO-containing EJ-309 and PPO-DIPN solution. Decreasing the amount of solubilizing agent IBA in the LSs (95:5 ratio) resulted in increasing the LO in each combination.

However, the most remarkable effect of ⁶LiDOSS loading is observed with PPO-containing EJ-309 that in this case loses only ˜10% of the LO compared to 30-45% observed with other methods of ⁶Li-loading. The loss of LO by the addition of ⁶Li salt was significantly less with the ⁶LiDOSS (FIG. 3A) compared to ⁶LiIVA (FIG. 4A).

FIG. 3B illustrates a preservation of the high PSD FoMs of each combination of EJ-309 with single dye and ⁶LiDOSS. The PSD FoM for three of ⁶LiDOSS loaded combination is above 3.9, reaching a record value of 4 in EJ-309 composition. Again, with no need for a solubilizing acid and due to the decreased polarity of ⁶Li in the molecule, the addition of ⁶LiDOSS produced less drop of PSD compared to other composition. For instance, FIG. 4B is a graph of the PSD FoM of EJ-309 and single dye solutions in DIPN in comparison to the aliphatic salt ⁶LiIVA-loaded solutions of the same compositions containing 10 wt. % and 5 wt. % of IBA as the solubilizing agent. The respective PSD FoMs measured at the energy of ¹³⁷Cs Compton edge for solutions without ⁶Li and the thermal energy spot position for all solutions containing 0.1 wt. % ⁶Li. None of these solutions shows FoM=4, having, like ⁶LiDOSS, LO only 10% below that of EJ-309. (FIG. 3B).

FIG. 5 is a plot of the dependence of ¹³⁷Cs LO (white circles, left y-axis) and ²⁵²Cf PSD FoM (solid circles, right y-axis) on ⁶Li concentration in solution containing 0.5 wt. % of E404 in combination with respective amounts of ⁶LiDOSS. Solutions proved to be stable to solid precipitation at ≤0.3 wt. % ⁶Li. In the absence of polar additions, ⁶LiDOSS-loaded EJ-309 also shows the highest FOM, largely determined by PSD properties of PPO less affected in this case by the ⁶Li addition. It should be added here that the high solubility of ⁶LiDOSS also allows for dissolution of larger concentrations of ⁶Li.

For comparison, FIG. 6 is a plot of the dependence of ¹³⁷Cs LO (white circles, left y-axis) and ²⁵²Cf PSD FoM (solid circles, right y-axis) on ⁶Li concentration (x-axis) in 90:10 DIPN:IBA solution containing 0.5 wt. % of E404 in combination with respective amounts of ⁶LiIVA. Results show that there is practically no change in high values of LO and PSD between 0.1 and 0.2 wt. % loads. Moreover, even at the maximum value of 0.5 wt. % ⁶Li, the total LO loss relative to unloaded EJ-309 does not exceed 20%, with still efficient PSD FOM of 2.5.

As shown in FIG. 5, in the case of ⁶LiDOSS loading, higher concentration of ⁶Li leads to the decrease of the LO that at 0.2 wt. % is about twice as high than in the case of ⁶LiIVA (FIG. 6). This larger drop can be attributed to a higher self-absorption or bigger dilution by the non-aromatic fraction of ⁶LiDOSS. However, the high performance combined with the simplicity of preparation that does not require addition of acids, which may cause long-term corrosive effects in large-volume liquid scintillators, makes ⁶LiDOSS-loaded EJ-309 one of the promising liquid scintillators for further development.

### Summary of ⁶Li-Loaded Liquid Scintillators: Small and Large Scale

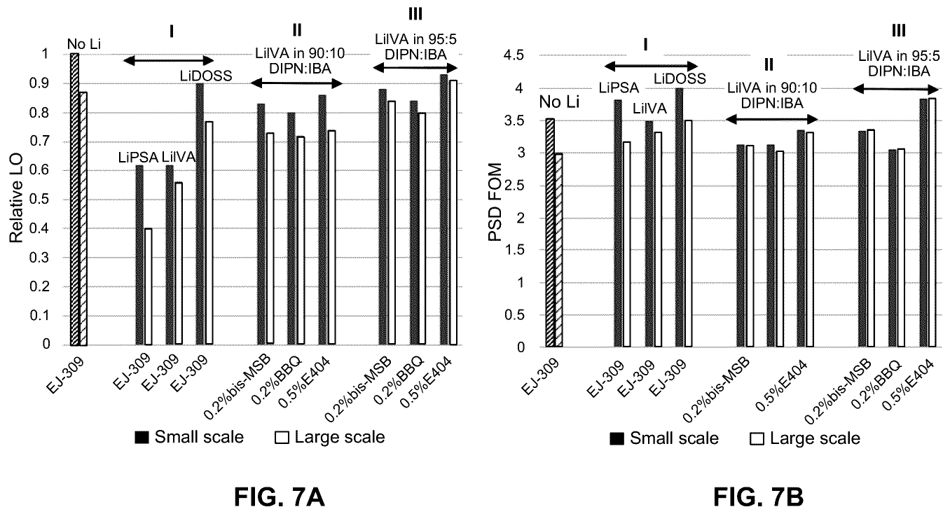

As illustrated in FIGS. 7A and 7B, a comparison of the most typical 0.1 wt. %-containing ⁶Li compositions obtained by dissolution of different compounds in EJ-309 or DIPN. FIG. 7A illustrates the relative LO measured for different ⁶Li-loaded formulations in comparison with the commercial, unloaded EJ-309. The ¹³⁷Cs LO results demonstrate a large variation of the LO loss in comparison with unloaded EJ-309. FIG. 7B illustrates the PSD measured, including the distribution of respective thermal neutron/gamma PSD FoMs, with different ⁶Li-loaded formulations in comparison with the commercial, unloaded EJ-309. The solid bars indicate small scale measurements made with (Ø2 cm×1 cm) quartz cuvettes, and white bars indicate large-scale measurements made with larger volume (Ø5 cm×5 cm) quartz cuvettes.

Considering that potential applications of ⁶Li-loaded LSs may involve large-volume preparations, each composition selected based on the small-scale (Ø2 cm×1 cm) measurements was additionally characterized with larger volume (Ø5 cm×5 cm) quartz cuvettes, for approximate evaluation of the self-absorption that produces negative effect on attenuation at increasing scale. The results were separated in the following three major groups characterized by the effects of ⁶Li-loading on scintillation performance in comparison to EJ-309 standard:

Group I: EJ-309 loaded with ⁶Li-organic salts. This group presents three compositions, among which loading with aromatic ⁶LiPSA and ⁶LiIVA produces the largest drop in the LO, compared to the initial EJ-309. The high degree of this drop (˜30-40%), that is close to that reported for the PROSPECT LS, can be attributed to possible reactions of polar ⁶Li and solubilizing acids with PPO that is a major constituent of EJ-309. Much smaller LO loss (˜10% relative to EJ-309) obtained with ⁶LiDOSS may result from decreased polarity and high solubility that does not require additions of water or acids. This makes ⁶LiDOSS-loaded EJ-309 LS more suitable for practical, large volume preparations, especially if such applications require high PSD facilitated by PPO. On a downside of this composition may be relatively high self-absorption that at larger scale may produce higher level of attenuation for both LO and PSD compared to the EJ-309 standard.

Group II: ⁶LiIVA-loading of single dye solutions in 90:10 DIPN:IBA liquid. These are non-PPO-containing solutions of very efficient single dyes, bis-MSB, BBQ, and E404. Due to the low dye concentrations (0.2 to 0.5 wt. %) they produce high LO that in ⁶Li-loaded versions may be more than 80% of EJ-309. LO attenuation may also be expected on the level of the unloaded EJ-309 standard. Solutions are highly stable, although some corrosion effects can be introduced by 10 wt. % of acid addition. A surprising result obtained with these compositions is low or no degradation of PSD measured with increasing sample size.

Group III: ⁶LiIVA-loading of single dye solutions in 95:5 DIPN:IBA liquid. This is an analog of group II that, due to the decreased addition of the IBA acid, produces highest LO of 92 to 84% relative to EJ-309 standard. Despite the lower concentration of IBA, no precipitation or LO degradation has been observed in any sealed samples over ˜1 year of observations. The group is characterized by the lowest self-absorption and no degradation of PSD measured with increasing sample size. However, the combination of the highest LO (90% of EJ-309) and the best FoM (4) is still shown by ⁶LiDOSS (FIGS. 7A and 7B).

### In Use

Various embodiments described herein may be developed for reactor antineutrino detection, antineutrino physics, advanced scintillation detection, etc.

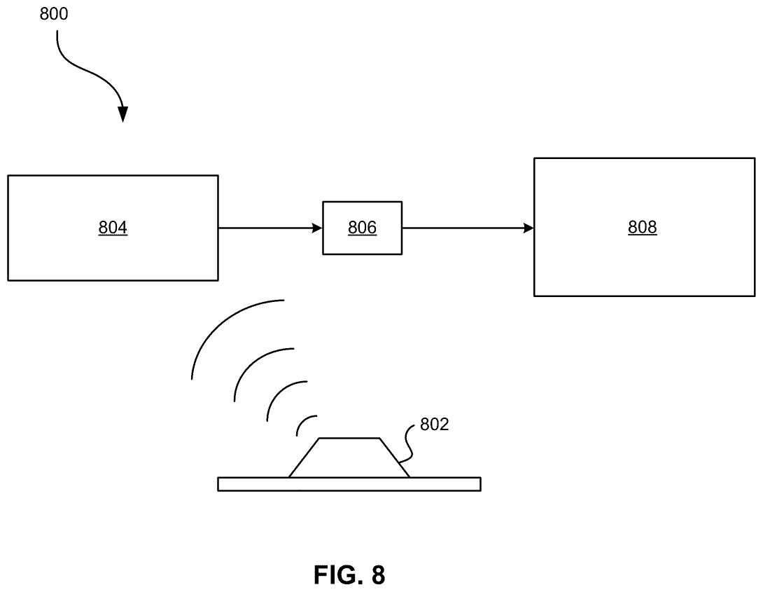

As described herein, one skilled in the art may use a scintillator material, a photodetector to detect the radiation-induced pulses and a device to translate the pulses to output discernable by humans. For example, FIG. 8 illustrates a simplified schematic of a detection system 800 according to one embodiment. As an option, the detection system 800 may be implemented in conjunction with features from any other embodiment listed herein, such as those described with reference to the other FIGS. Of course, the detection system 800 and others described herein may be used in various applications and/or in permutations which may or may not be specifically described in the illustrative embodiments listed herein. For instance, the detection system 800 may include more or less components than those shown in FIG. 8, in various approaches.

As shown in FIG. 8, the detection system 800 comprises a scintillator material 802, such as of a type described herein, and which is referred to herein interchangeably as a scintillator. The system 800 also includes a photodetector 804, such as a photomultiplier tube, a silicon photomultiplier, photodiode, or other device/transducer known in the art, which can detect and register the magnitude of the light emitted from the scintillator 802. The detection system 800 is preferably configured to register x-rays, gamma rays, and fast or thermal neutrons, as well as being able to distinguish between said forms of radiation.

The scintillator 802 produces light pulses upon occurrence of an event, such as a neutron, a gamma ray, an x-ray, or other radiation engaging the scintillator 802. For instance, as a gamma ray traverses the scintillator 802, photons are released, appearing as light pulses emitted from the scintillator 802. The light pulses are detected by the photodetector 804 and transduced into electrical signals that correspond to the magnitude of the pulses. The type of radiation can then be determined by analyzing the integrals of the light pulses and thereby identifying and separating the signals produced by neutron or gamma rays in the scintillator.

In some embodiments, the system 800 may be, further comprise, or be coupleable/coupled to, a preamplifier and/or digitizer (not shown in FIG. 8).

In other embodiments, the system 800 may include a processing device 806 configured to process pulse traces output by the photodetector 804, which correspond to light pulses from the scintillator 802. In some approaches, the processing device 806 may be further configured to generate radiological image data based on the pulse traces output by the photodetector 804.

In additional approaches, system 800 may include a processing device that receives data from a photodetector that is not permanently coupled to the processing device. Illustrative processing devices include microprocessors, field programmable gate arrays (FPGAs), application specific integrated circuits (ASICs), computers, etc.

The result of the processing may be output and/or stored. For example, the result may be displayed on a display device 808 in any form, such as in a histogram of the number of counts received against the total light from the scintillator or derivative thereof.

The program environment in which one embodiment of the invention may be executed illustratively incorporates one or more general-purpose computers or special-purpose devices such hand-held computers. Details of such devices (e.g., processor, memory, data storage, input and output devices) are well known and are omitted for the sake of clarity.

It should also be understood that the techniques of the present invention might be implemented using a variety of technologies. For example, the methods described herein may be implemented in software running on a computer system, or implemented in hardware utilizing one or more processors and logic (hardware and/or software) for performing operations of the method, application specific integrated circuits, programmable logic devices such as Field Programmable Gate Arrays (FPGAs), and/or various combinations thereof. In particular, methods described herein may be implemented by a series of computer-executable instructions residing on a storage medium such as a physical (e.g., non-transitory) computer-readable medium. In addition, although specific embodiments of the invention may employ object-oriented software programming concepts, the invention is not so limited and is easily adapted to employ other forms of directing the operation of a computer.

Portions of the invention can also be provided in the form of a computer program product comprising a physical computer readable medium having computer code thereon. A computer readable medium can include any physical medium capable of storing computer code thereon for use by a computer, including optical media such as read only and writeable CD and DVD, magnetic memory or medium (e.g., hard disk drive), semiconductor memory (e.g., FLASH memory and other portable memory cards, etc.), etc.

The inventive aspects disclosed herein have been presented by way of example to illustrate the myriad features thereof in a plurality of illustrative scenarios, aspects of an inventive aspect, and/or implementations. It should be appreciated that the aspects generally disclosed are to be considered as modular, and may be implemented in any combination, permutation, or synthesis thereof. In addition, any modification, alteration, or equivalent of the presently disclosed features, functions, and aspects that would be appreciated by a person having ordinary skill in the art upon reading the instant descriptions should also be considered within the scope of this disclosure.

While various aspects of an inventive aspect have been described above, it should be understood that they have been presented by way of example only, and not limitation. Thus, the breadth and scope of an aspect of an inventive aspect of the present invention should not be limited by any of the above-described exemplary aspects of an inventive aspect, but should be defined only in accordance with the following claims and their equivalents.

What is claimed is:

1. A lithium (Li)-containing compound, wherein the Li-containing compound is a lipophilic Li salt,
wherein each molecule of the lipophilic Li salt has at least one lipophilic endgroup.

2. A Li-containing compound as recited in claim 1, wherein the at least one lipophilic end group is selected from the group consisting of: a polycyclic aromatic hydrocarbon, an octyl succinate, an alkylsuccinate, an alkylsulfosuccinate, and a petroleum sulfonate.

3. A Li-containing compound as recited in claim 1, wherein the lipophilic Li salt is soluble in an organic liquid in a range of greater than 1 gram of the lipophilic Li salt per 100 milliliters of the organic liquid up to 100 grams of the lipophilic Li salt per 100 milliliters of the organic liquid.

4. A Li-containing compound as recited in claim 1, wherein the lipophilic Li salt is a surfactant.

5. A Li-containing compound as recited in claim 1, wherein the lipophilic Li salt is Li 1,4-bis(2-ethylhexoxy)-1,4-dioxobutane-2-sulfonate (⁶LiDOSS).

6. A scintillator material comprising an organic liquid and the Li-containing compound of claim 1, wherein the Li-containing compound is solubilized in the organic liquid.

7. A scintillator material as recited in claim 6, wherein a solubilizing agent is not present in the scintillator material.

8. A scintillator material as recited in claim 6, wherein the organic liquid includes an aromatic compound.

9. A scintillator material as recited in claim 6, wherein the organic liquid is configured to be cured into a plastic matrix material.

10. A scintillator material, comprising a plastic matrix material constructed, at least in part, of a cured organic liquid and the Li-containing compound of claim 1.

11. A scintillator material, comprising:
an organic liquid; and
a lipophilic lithium (Li) salt, wherein the lipophilic Li salt is solubilized in the organic liquid,
wherein the scintillator material includes at least one fluorescent dye, the dye being effective to provide scintillation upon exposure to radiation,
wherein the scintillator material exhibits an optical response signature for thermal neutrons that is different than an optical response signature for fast neutrons.

12. A scintillator material as recited in claim 11, wherein a solubilizing agent is not present in the scintillator material.

13. A scintillator material as recited in claim 11, wherein a polar liquid is not present in the scintillator material.

14. A scintillator material as recited in claim 11, wherein the organic liquid is selected from the group consisting of: xylene, pseudocumene, toluene, styrene, EJ-309, alkene benzene, and 2,6-diisopropylnaphthalene.

15. A scintillator material as recited in claim 11, wherein the organic liquid is a scintillator.

16. A scintillator material as recited in claim 11, wherein the lipophilic Li salt is a surfactant, wherein a second surfactant is not present in the scintillator material.

17. A scintillator material as recited in claim 11, wherein the scintillator material is a liquid scintillator.

18. A scintillator material as recited in claim 11, wherein only a single fluorescent dye is present in the scintillator material.

19. A scintillator material as recited in claim 11, wherein the scintillator material is configured to have a relative light output upon scintillation that is at least 85% of the light output of the scintillator material without the Li-containing compound.

20. A plastic scintillator, comprising:
a polymer matrix; and
a lipophilic lithium (Li) salt, wherein the lipophilic lithium salt is solubilized in the polymer matrix,
wherein the plastic scintillator includes at least one fluorescent dye, the dye being effective to provide scintillation upon exposure to radiation,
wherein the plastic scintillator exhibits an optical response signature for thermal neutrons that is different than an optical response signature for fast neutrons.
